(19) United States
(12) Patent Application Publication
(10) Pub. No.: US 2006/0089308 A1
(43) Pub. Date: Apr. 27, 2006

(54) Method of improving ovulation induction using an androgen such as dehydroepiandrosterone

(75) Inventors: Norbert Gleicher, Chicago, IL (US); David H. Barad, Closter, NJ (US); Dwyn V. Harben, Bryn Mawr, PA (US)

Correspondence Address: BEEM PATENT LAW FIRM, 53 W. JACKSON BLVD., SUITE 1352, CHICAGO, IL, 60604-3787, US

(73) Assignee: American Infertility of New York

(21) Appl. No.: 10/973,192

(22) Filed: Oct. 26, 2004

(51) Int. Cl. A61K 38/09; A61K 31/57

(52) U.S. Cl. 514/15; 514/171

(57) ABSTRACT

A method of preconditioning ovulation induction in a human female comprises of administering an androgen, for example, DHEA, for at least about four consecutive months. DHEA may be administered along with high dose gonadotrophins in ovulation induction treatments. Moreover, DHEA may be administered with follicle stimulating hormone, human menopausal gonadotrophin, norethindrone acetate, leuprolide acetate, and human chorionic gonadotrophin in ovulation induction treatments.

### BACKGROUND OF THE INVENTION

1. Field of the Invention

The present invention relates to a method of improving ovulation induction in women undergoing in vitro fertilization and other infertility treatments involving ovarian stimulation by administering an androgen such as dehydroepiandrosterone prior to or during ovulation stimulation cycles.

2. Description of the Related Art

The application of assisted reproductive technology has revolutionized the treatment of all forms of infertility. The most common assisted reproductive technology is in vitro fertilization (IVF), in which a woman's eggs are harvested and fertilized with a man's sperm in a laboratory. Embryos grown from the sperm and eggs are then chosen to be transferred into the woman's uterus. Assisted reproductive technology in women depends on ovarian stimulation and concurrent multiple oocyte development, induced by exogenous gonadotrophins.

Infertile women are often treated with gonadotrophin treatments such as gonadotrophin-releasing hormone (GnRH) flare protocols. Estrogen pre-treatment with concomitant growth hormone (GH) treatment is sometimes used in an effort to try and amplify intra-ovarian insulin-like growth factor-I (IGF-I) paracrine effect, which is expressed by granulosa cells and enhances gonadotrophin action. However, the clinical utility of combined GH/ovarian stimulation is limited and responses are not dramatic.

Dehydroepiandrosterone (DHEA) is secreted by the adrenal cortex, central nervous system and the ovarian theca cells and is converted in peripheral tissue to more active forms of androgen or estrogen. DHEA secretion during childhood is minimal but it increases at adrenarche and peaks around age 25, the age of maximum fertility, only to reach a nadir after age 60. There is evidence to support use of exogenous DHEA to increase ovulation stimulation in older women who respond poorly to gonadotrophin treatments. First, studies demonstrate marked augmentation of serum IGF-I concentrations of oral administration of physiological DHEA. Second, DHEA is a steroid prohormone for ovarian follicular sex steroidogenesis.

Third, Casson studies have shown that concurrent oral DHEA supplementation over about two months and one or two stimulation cycles improved gonadotrophin response by approximately two-fold in women who had normal follicular stimulating hormone concentrations, yet had poor response to ovarian stimulation. Frattarelli and Peterson found that cycle day 3 testosterone above 20 ng/dL was associated with higher IVF pregnancy rates (11.2% vs. 53.1%). Approximately 25 to 50 mg of DHEA is considered physiologic replacement for young females. Adverse effects are extremely uncommon at such dosages, while dosages as high as 1600 mg daily have caused significant side effects, requiring discontinuation of treatment.

The “aging ovary” represents the last frontier of human infertility treatment and is generally considered untreatable with current medical resources. The possibility that any intervention may significantly benefit the response of the aging ovary is therefore potentially revolutionary. The studies show many ways in which ovulation induction can be improved in infertile women. These studies show DHEA as possible, but not preferred, treatments for improving ovulation induction.

### BRIEF SUMMARY OF THE INVENTION

The present invention is directed to the administration of an androgen for at least about four consecutive months, to precondition ovulation induction in women. In one embodiment, the androgen is dehydroepiandrosterone (DHEA). DHEA administration may be conducted orally in patients. In conjunction with DHEA, high dose gonadotrophins may be administered. Also in conjunction with DHEA, follicle stimulating hormone (FSH), norethindrone acetate, leuprolide acetate, and gonadotrophin may be used to maximize ovulation induction.

In a further aspect, the invention relates to the administration of an androgen for at least about five and a half consecutive months, to precondition ovulation stimulation in a woman. In one embodiment the androgen comprises DHEA.

### BRIEF DESCRIPTION OF THE DRAWINGS

FIG. 1 is a table showing improved ovulation induction with DHEA.

FIG. 2 is a graph showing increase in production of oocytes and cryopreservable embryos over DHEA treatment cycles.

### DETAILED DESCRIPTION OF THE INVENTION

When attempting in vitro fertilization, older women produce few oocytes and yield few normal embryos, even when exposed to maximal gonadotrophin stimulation. The decreased ability of older women to respond to ovulation inducing medications is evidence that ovarian reserve declines with age. Treatments with an androgen, alone or in conjunction with other hormones, increase a woman's response to ovulation induction, measured in both oocyte and embryo yield. Androgens may be, for example, dehydroepiandrosterone (DHEA) or testosterone. DHEA treatment is an adjunct to ovulation induction. DHEA taken orally for about four months before initiating gonadotrophin treatment may prepare the ovaries for gonadotrophin stimulation. It is believed that a larger response may be obtainable by combining gonadotrophins and DHEA in treatment over about a four month period before an IVF cycle.

Young ovaries are characterized by large numbers of antral follicles and a low rate of atresia. In contrast, older ovaries have few antral follicles, high rates of atresia and exhibit increasing “resistance” to ovulation induction. With IVF, older women have decreased oocyte quantity and quality, produce fewer high quality embryos and have lower implantation and pregnancy rates. Most follicular atresia occurs after the primordial follicle resumes growth but before it is gonadotrophin responsive enough for recruitment. An induced delay in onset of atresia may salvage follicles for possible ovulation. Interestingly, such an “arrest” of the atretic process has been noted among anovulatory women with polycystic ovary syndrome (PCO). For these women follicles remain steroidogenicaly competent and show evidence of increased aromatase activity compared to like-sized follicles from normal ovaries. Follicular hypersecretion of DHEA, which is typical of PCO, is associated with increased aromatase activity. The increased yield of oocytes and embryos experienced by patients undergoing DHEA treatment also suggest this underlying physiological process.

Possible side effects associated with DHEA use are acne, deepening voice and facial hair growth, though long-term effects of DHEA administration are unknown. As a precursor of sex steroids one, of course, has to be concerned abut the potential effect on hormone-sensitive malignancies.

### EXAMPLE 1

A 43 year old woman undergoing IVF with banking of multiple cryopreserved embryos for future aneuploidy screen and transfer is administered an androgen, namely DHEA. In ten months she undergoes eight treatment stimulation cycles while continuously improving her ovarian response, resulting in oocyte and embryo yields far beyond those previously seen in a woman her age.

The patient's history is unremarkable except for two previous malarial infections. She is allergic to sulfa mediations and has a history of environmental allergies. Her surgical history includes umbilical hernia repair at age one and cholecystectomy at age 21. She had used oral contraceptives for over 10 years. She has no history of irregular menstrual cycles.

Day three serum FSH and estradiol (E2) in her first IVF cycle are 11 mIU/ml and 18 pg/ml, respectively. In subsequent cycles her baseline FSH is as high as 15 mIU/ml. She is given an ovulation induction protocol which is prescribed for patients with evidence of decreased ovarian reserve. Briefly, the protocol includes the following medications: norethindrone acetate tablets (10 mg) for 10 days, starting on day two of menses, followed three days later by a “microdose” dosage of 40 μg of leuprolide acetate, twice daily, and, after another three days, by 600 IU of FSH (Gonal-F; Ares-Serono, Geneva, Switzerland) daily. Peak serum E2 concentration on day nine of stimulation is 330 pg/ml. Following injection of 10,000 IU human chorionic gonadotrophin (hCG), she undergoes oocyte retrieval. Only one oocyte is obtained and one embryo is cryopreserved.

Because of the poor response to ovulation stimulation, she is advised to consider donor oocyte or embryo donation. She rejects both options. She starts a second cycle using the same stimulation protocol with one exception: instead of 600 IU of FHS daily, the patient received 450 IU of FSH and 150 IU of human menopausal gonadotrophin (HMG, Pergonal, Ares-Serono, Geneva, Switzerland). This stimulation protocol is continued in identical fashion for the remaining cycles. However, two weeks before starting her second cycle, she begins administration of 75 mg per day of oral micronized DHEA. The date on which she begins administration of 75 mg per day of oral micronized DHEA is Oct. 6, 2003.

Methods

The eight treatment cycles is divided into three groups to allow statistical comparison: pre-initiation and very early use of DHEA (early=cycles 1 and 2), initial cycles (mid=cycles 3-5), and later cycles (late=cycles 6-8). Comparison between these categories is by one-way analysis of variance (ANOVA) and multiple comparisons by Student-Neuman-Keuls (SNK) test. The homogeneity of variances and used orthogonal linear contrasts are tested to compare groups and polynomial contrast to test for linear and quadratic trends. All outcomes are presented as mean±1 standard deviation. Rate of change of oocyte counts, cryopreserved embryos and (log transformed) peak estradiol between subsequent cycles is estimated by linear regression.

Embryos are evaluated by the embryologists on day three post-insemination for cell-count and grading. Embryo grading is based on a 1 to 4 scale depending on symmetry, percent fragmentation and appearance of the cytoplasm. All viable embryos are cryopreserved. Statistics are performed using SPSS for Windows, Standard version 10.0.7 (SPSS Co., Chicago, Ill.). Assay of E2 and FSH are performed using the ACS: 180 chemoluminescence system (Bayer Health Care LLC, Tarrytown, N.Y.).

A method of preconditioning ovulation induction in a human female is conceived, comprising administering an androgen in a female for at least about four consecutive months. In one embodiment, the androgen is DHEA. Administration of DHEA for at least about four consecutive months may further comprise administering high dose gonadotrophins to the female. Furthermore, DHEA may be administered along with follicle stimulating hormone, human menopausal gonadotrophin, norethindrone acetate, leuprolide acetate, and human chorionic gonadotrophin. DHEA may be administered orally.

The length of time the androgen is administered to the female can also be more than five and a half consecutive months. In one embodiment, the androgen administered is DHEA.

Results

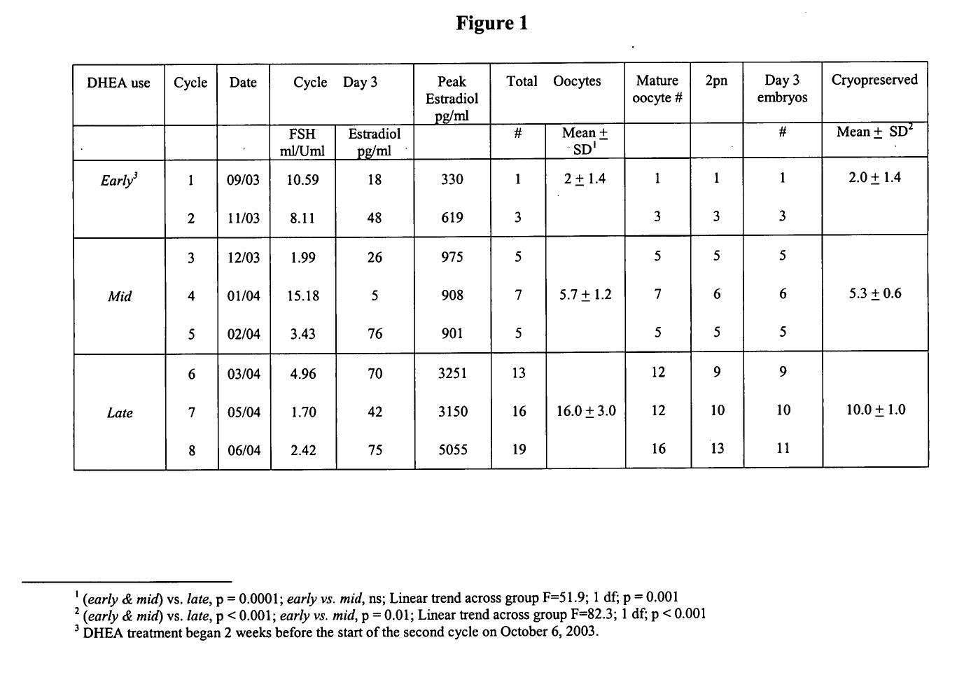

The results of ovulation induction are displayed in FIG. 1. After eight stimulation cycles and approximately eight months of DHEA treatment, the patient produced 19 oocytes and 11 cryopreservable embryos. A total of 50 viable embryos have so far been cryopreserved. Significantly more oocytes (p=0.001) and cryopreserved embryos (p<0.001) are obtained in the late cycles (cycles 6-8, 4+consecutive months of DHEA treatment) compared to the combined early and mid cycles (cycles 1-5, 0-4 consecutive months of DHEA treatment). There is no significant difference in average embryo cell count (6.83±1.37 vs. 7.2±1.15) or morphology (3.6±0.5 vs. 3.7±0.5) between early and mid compared to late cycles. Peak E2, total oocyte, and embryos cryopreserved increase linearly from cycle to cycle, as shown in FIG. 1. Oocyte yield increase 2.5±0.34 oocytes per cycle (p<0.001), cryopreservable embryo yield increase 1.4±0.14 embryos per cycle (p<0.001) and (log) peak E2 increase 0.47±0.06 (p<0.001) across treatment cycles.

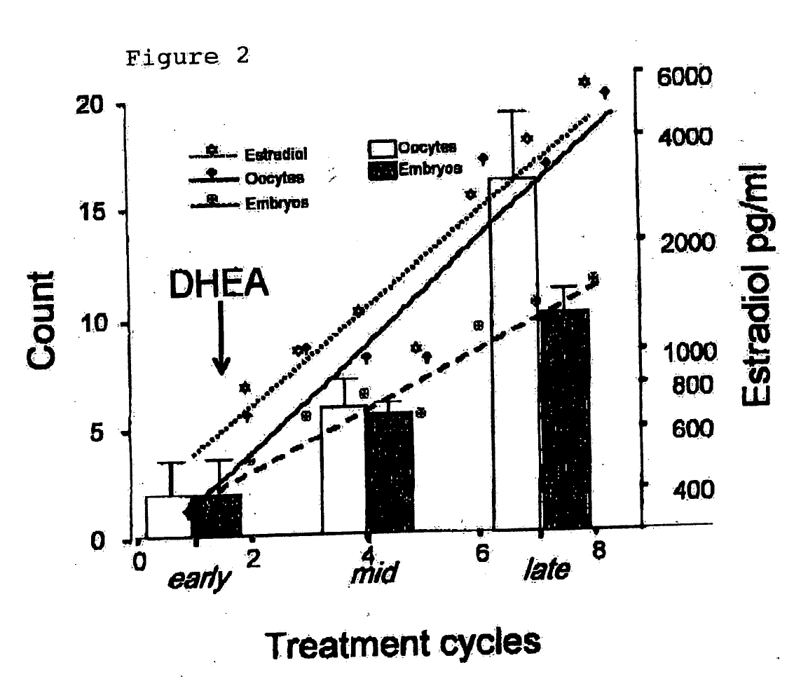

The linear increase in (log) peak E2 shown in FIG. 2 represents a cycle to cycle rate of increase from 123 pg/ml/cycle to 1491 pg/ml/cycle over the eight cycles of treatment. After adjusting for cycle day, the (harmonic) mean E2 is 267 pg/ml (95% confidence intervals (CI) 143 to 498 pg/ml) in the early phase, 941 pg/ml (95% CI 518 to 1712 pg/ml) in the mid phase, and 1780 pg/ml (95% CI 1121 to 2827 pg/ml) in the late phase of treatment. Each of these homogeneous subsets is significantly different from the other (p<0.05) by SNK multiple comparison testing.

The dramatic increase in oocyte and embryo yield experienced by this 43 year old woman is completely surprising and unexpected. The patient's post-DHEA response to ovulation induction has become more like that of a younger woman with PCO, than that of a 43 year old woman. Since starting DHEA treatment, the patient has produced 49 embryos of high enough quality to undergo cryopreservation. Sixty percent of those embryos were produced in the last three cycles of treatment, which took place after at least about four consecutive months after starting treatment. After producing only one embryo prior to starting DHEA treatment, the patient improved by an order of magnitude and produced 13 oocytes and 9 embryos in a cycle after at least about four consecutive months of DHEA treatment, 16 oocytes and 10 embryos in a cycle after at least about five and a half consecutive months of DHEA treatment, and 19 oocytes and 11 embryos in a cycle after at least about seven consecutive months of DHEA treatment. The increasing numbers of cryopreservable embryos may suggest that embryo quality has improved. Quantity of embryos definitely is improved and quality may be improved.

This patient took DHEA supplementation along with high dose gonadotrophins for several months. It is believed that her response may represent an interaction of these treatments.

The preceding example is to be construed as merely illustrative and not limitative of the remainder of the disclosure in any way.

What is claimed is:

1. A method of preconditioning ovulation induction in a human female comprising administering an androgen in said female for at least about four consecutive months.

2. A method according to claim 1, wherein said androgen comprises dehydroepiandrosterone.

3. A method according to claim 2, further comprising administering high dose gonadotrophins.

4. A method according to claim 2, further comprising administering follicle stimulating hormone, human menopausal gonadotrophin, norethindrone acetate, leuprolide acetate, and human chorionic gonadotrophin.

5. A method according to claim 2, wherein said administering step is conducted orally.

6. A method of preconditioning ovulation induction in a human female comprising administering an androgen in said female for at least about five and a half consecutive months.

7. A method according to claim 6, wherein said androgen comprises dehydroepiandrosterone.
